(19) United States
(12) Patent Application Publication
(10) Pub. No.: US 2006/0004189 A1
(43) Pub. Date: Jan. 5, 2006

(54) Compositions for treating wounds and processes for their preparation

(76) Inventor: James Gandy, Calhoun, LA (US)

Correspondence Address: Warren K. MacRae;Bryan Cave LLP, 1290 Avenue of the Americas, New York, NY, 10104-3300, US

(21) Appl. No.: 11/173,340

(22) Filed: Jul. 1, 2005

Related U.S. Application Data
(60) Provisional application No. 60/585,403, filed on Jul. 2, 2004.

(51) Int. Cl. C07K 14/475

(52) U.S. Cl. 530/399

(57) ABSTRACT

The present invention provides a process for obtaining growth factors by treating a growth factor starting material to release growth factors from the growth factor starting material and recovering growth factors from the treated growth factor starting material. The growth factor obtained can be used in compositions to treat humans.

### CROSS REFERENCE TO RELATED APPLICATIONS

This is a continuation of Provisional Patent Application Ser. No. 60/585,403, filed Jul. 02, 2004. All priority rights to and for that application are hereby claimed.

### BACKGROUND OF THE INVENTION

Platelet Rich Plasma (PRP) is a by-product of blood (plasma) that is rich in platelets. Due mainly to the cost of separating the platelets from the blood and the large amount of blood needed (one unit) to produce a suitable quantity of platelets, its use has until recently been confined to the hospital setting or blood bank. New technology permits the doctor to harvest and produce a sufficient quantity of platelets from only 55 cc of blood drawn from a patient while the patient is having outpatient surgery.

Platelet Rich Plasma permits the body to take advantage of the normal healing pathways at a greatly accelerated rate. During the healing process, the body rushes many cells and cell-types to the area of insult, such as a wound, in order to initiate the healing process. One of those cell types is platelets. Platelets perform many functions, including formation of a blood clot and release of growth factors (GF) into the area of insult. Growth factors are peptides that act on inflammatory cells, fibroblasts, and endothelial cells to direct the processes involved in wound healing. They are present immediately after an insult because platelet derived growth factors (PDGF) and basic fibroblast growth factor (bFGF) are produced by the cells at the time of injury. Subsequently, activated platelets release transforming growth factor beta (TGF-beta) and PDGF to mediate chemotaxis of neutrophils, monocytes, and fibroblasts into the wound.

Additionally, the injured tissue locally releases eicosanoids, which amplify the early response to injury. Eicosanoids are arachidonic acid metabolites that are derived from cell membrane fatty acids. Activated phospholipase A catalyzes the production of prostaglandins and thromboxane from the arachidonic acid. These substances play central roles in the regulation of vasomotor and platelet activity after injury. Thromboxane A2 helps with hemostasis by its effects of vasoconstriction and platelet aggregation.

These growth factors (platelet derived growth factors PGDF, transforming growth factor beta TGF-beta, and insulin-like growth factor ILGF) function to assist the body in repairing itself by stimulating stem cells to regenerate new tissue. The more growth factors released and sequestered into the wound, the more stem cells stimulated to produce new host tissue. Thus, PRP permits the body to heal faster and more efficiently.

A subfamily of TGF, is bone morphogenic protein (BMP). BMP has been shown to induce the formation of new bone in research studies in animals and humans. This is of great significance to the surgeon who places dental implants. By adding PRP, and thus BMP, to the implant site with bone substitute particles, the implant surgeon can now cause the patient's body to grow bone more predictably and faster than ever before.

PRP has many clinical applications:
Bone grafting for dental implants. This includes onlay and inlay grafts, sinus lift procedures, ridge augmentation procedures, and closure of cleft, lip and palate defects.
Repair of bone defects creating by removal of teeth or small cysts.
Repair of fistulas between the sinus cavity and mouth.
PRP also has several safety advantages. For example, since PRP is generally a by-product of the patient's own blood, disease transmission is not an issue.
Convenience: PRP can be generated in the doctor's office while the patient is undergoing an outpatient surgical procedure, such as placement of dental implants.
Faster healing: The super saturation of the wound with PRP, and thus growth factors, produces an increase of tissue synthesis and thus faster tissue regeneration.
Cost effectiveness: Since PRP harvesting can be done with only 55 cc of blood in the doctor's office, the patient need not incur the expense of the harvesting procedure in hospital or at the blood bank.
Ease of use: PRP is easy to handle and actually improves the ease of application of bone substitute materials and bone grafting products by making them more gel-like.

Knighton, U.S. Pat. No. 4,957,742, discloses platelet enriched plasma produced from blood wherein the platelets are activated by thrombin which causes the release of platelet-derived growth and angiogenesis factors. A carrier, such as a microcrystalline collagen, is added to produce a wound-treating salve, and the composition is applied directly to wounds and initiates healing in nonhealing wounds as well as accelerating normal wound-healing by increasing vascularization, stimulating fibroblast mitosis and migration, and increasing collagen synthesis by fibroblasts. It is said that the composition may also be applied to tissue to facilitate the growth of hair.

Worden, U.S. Pat. No. 6,524,568, discloses a platelet gel wound healing composition that includes growth factors and ascorbic acid and optionally including an anti-oxidant such as Vitamin A and/or Vitamin E. Antibiotics may also be included.

Chao, U.S. Pat. No. 5,185,160, discloses a heat-treated, viral-inactivated platelet membrane microparticle fraction which may be prepared from outdated platelets. The microparticle fraction is said to be substantially free of platelet ghosts and may be used as a pharmaceutical preparation in transfusions.

Chao, U.S. Pat. No. 5,332,578, also discloses a heat-treated, viral-inactivated platelet membrane microparticle product which may be prepared from outdated mammalian platelets. The microparticle product is said to contain isolated platelet membrane fragments that are free of alloantigens and GP IIb/IIIa complexes and it is said that the product may be used as a pharmaceutical preparation in transfusions.

Crowe, U.S. Patent Application Publication No. U.S. 2004/0265293A1, discloses a dehydrated composition that includes freeze-dried platelets. The platelets are loaded with trehalose in an amount from about 10 mM to about 50 mM, and at a temperature of from greater than about 25° C. to less than about 40° C. The freeze-dried platelets are said to be substantially shelf-stable and are rehydratable so as to have a normal response to an agonist, for example, thrombin, and it is said that virtually all of the platelets participate in clot formation within about three minutes at 37° C.

Van der Meulen, et al., Isolation and Partial Characterization of Platelet α-Granule Membranes, J. Membrane Biol. 71, 47-59 (1983), discloses porcine α-granules that were found to be essentially homogeneous by transmission electron microscopy. Freeze-fractured samples of isolated granules revealed intramembranous particles on the exoplasmic fracture surface and, to a lesser extent, on the protoplasmic fracture surface, whereas the PS (protoplasmic) surface was relatively smooth and, it is said, the granules appeared to be sealed. Membranes were isolated by alkali extraction of the granules which removed protein and phospholipids yielding membrane vesicles devoid of the dense core. The membranes were said to contain major and minor polypeptides. The exposure of specific proteins on the cytoplasmic surface of the granule membrane was also determined. In sealed granules, bands were modified by the reagents, and a fraction eluted by alkali extraction was also analyzed and found to contain nine major polypeptides.

Chao, et al., Infusible platelet membrane microvesicles: potential transfusion substitute for platelets, transfusion, 36:536-542, (1996), discloses preparation of IPM from outdated platelets. The platelets were disrupted by freezing and thawing, washed and heated to inactivate possible viral contaminants, and then a sonicated membrane microvesicle fraction was separated and lyophilized. The hemostatic activity of IPM was measured by its ability to reduce the prolonged bleeding time in thrombocytopenic rabbits.

According to Chao, administration of IPM at a dose of 2 mg per kg results in a substantial reduction in the bleeding time. It is reported that, in a series of 23 experiments, a median preinjection bleeding time of 15 minutes was reduced to 6 minutes within 4 hours after IPM administration. Administration of IPM was said to show a mild enhancement in the thrombogenicity index, as measured in the Wessler rabbit model, which was not significant. Chao concludes that IPM may have clinical potential as a substitute for platelets in the treatment of bleeding due to thrombocytopenia.

Gogstad, A Method For The Isolation Of α-Granules From Human Platelets, Thrombosis Research, 20:669-681 (1980), discloses a method for the isolation of α-granules wherein a two-step French pressure cell homogenization procedure produced an organelle concentrate for loading on density gradients. The procedure was said to be optimalized with respect to recovery of intact α-granules. The organelle homogenate was loaded to 17.5-27.5% metrizamide gradients and centrifuged. Organelle aggregate formation was said to be minimized by controlling the ionic conditions and the shape of the gradient. The α-granules were separated from lysosomes and dense bodies, but overlapped with the mitochondria, and the α-granules were recovered from the gradient to omit the major amount of mitochondria from the final preparation.

Hernandez, In Vitro Evaluation of the Hemostatic Effectiveness of Non Viable Platelet Preparations: Studies with Frozen-Thawed, Sonicated or Lyophilized Platelets, Vox Sang 73:36-42 (1997), discloses an investigation into the effects on hemostatsis of nonliving platelet derivatives. The effects of different platelet preparations on primary hemostatsis in a well-established perfusion model were evaluated, and studies were carried out with blood anticoagulated with low molecular weight heparin. Frozen-thawed, sonicated or lyophilized platelets were added to normal blood or to blood which had been experimentally depleted of platelets. Platelet interaction with the subendothelium and fibrin deposition were morphometrically evaluated. Hernandez reports that addition of nonviable platelet preparations to thrombocytopenic blood promoted a statistically significant increase in the deposition of fibrin on the subendothelium, but only lyophilized platelets retained some ability to interact with the subendothelium. Flow cytometry studies demonstrated the presence of BPIb, GPIIIa and P-selection on lyophilized platelets. Hernandez concludes that preparations containing nonviable platelets may still retain some hemostatic properties.

### SUMMARY OF THE INVENTIONS

The present invention provides compositions for promoting stimulation and growth of tissues. More particularly, the compositions include growth factors which may be isolated and purified or substantially purified. The compositions may be used to treat insults to the body, such as burns, cuts, and scrapes, contusions, including oral and otolaryngological wounds, wounds that are caused and treated by plastic surgery, and bone damage.

The compositions may used alone or in combination therapy together with other growth promoting actives, such as isolated and purified or synthetically produced protein compounds, and/or pain and inflammation reducing factors.

The present invention also provides processes for obtaining growth factors; for preparing growth factor-containing compositions; and methods of using the compositions prepared, alone or in combination therapy. The compositions of the invention may be prepared by treating a growth factor starting material. Growth factors may then be recovered from the treated growth factor starting material. The compositions of the invention may be administered to a patient in need thereof in an amount effective to treat a wound. Thus, the present invention also provides methods of treating patients by administering to a patient in need thereof an effective amount of a composition according to the invention as described herein.

The present invention also provides a kit that includes elements for preparing a wound treating composition. The kit may include an amount of the composition contained in a form to be applied to a wound applicator element for applying the composition to a wound.

### DETAILED DESCRIPTION OF THE INVENTIONS

The compositions of the present invention can promote stimulation and growth of tissues including epithelial tissue, which further includes simple or stratified squamous, cuboidal and columnar epithelial tissue; connective tissue such as loose or dense, cartilage, adipose, bone, and blood connective tissue; muscle tissue such as voluntary and involuntary, striated and smooth, and cardiac muscle tissue; and nervous tissue such as central nervous system (CNS) tissue, which is comprised of the brain and spinal cord, and the peripheral nervous system (PNS) tissue, which is comprised of all the other nervous tissue in the body. The composition may also be used for organ regeneration, reducing scaring, for cosmetic applications, such as, cosmetic surgery, treating sun-damaged skin, wrinkles, promoting hair growth, as a haemostatic agent, or as a medium for growth of cells and cultures. As used herein, the term “composition” is intended to mean a plurality of growth factors of one or more types alone or in combination with one or more of the other elements described below. The compositions may contain growth factors in isolated and/or purified form, and the growth factors of the composition may be in, or include, synthetic form. It is also believed that addition of stem cells will aid in the healing process. As used herein, percentages are based on the weight of the composition.

The compositions may include from about 0.1% to about 99.9% growth factors. In terms of nanograms per milliliters, they will include from about 10 ng/ml to about 500 ng/ml growth factors, more preferably from about 40 ng/ml to about 300 ng/ml growth factors, and most preferably from about 90 ng/ml to about 220 ng/ml growth factors. Preferably, the growth factors will be platelet derived growth factors (PDGF).

When a composition of the invention includes a reduced amount of growth factors, it is beneficial to increase the amount of plasma, preferably containing epidermal growth factor (“EGF”), or simply an amount of EGF, that the composition can contain. This can be accomplished by including a predetermined amount of plasma in the original platelet rich plasma or by adding plasma to the growth factors obtained by the processes of the invention.

Thus, an example of a composition of the invention includes a minor amount of growth factors, such as from about 0.1% to about 10%, and a major amount of plasma, such as from about 90% to about 99.9% plasma. Another composition may include about 25% growth factors and about 75% plasma. Alternatively, an example of a composition according to the invention may include major amount of growth factors and a minor amount of plasma, such as about 90% growth factors and about 10% plasma, or about 75% growth factors and about 25% plasma, or about 50% growth factors and 50% plasma.

Another example of a composition of the invention would include an amount of EGF that approaches or is the same as the amount of EGF that is naturally found in a sample of blood plasma in a normal human, depending, of course, on the particular age (e.g., child or adult). Alternatively, a composition of the invention may include a minor amount of growth factors, such as from about 0.1% to about 10%, and a major amount of EGF, such as from about 90% to about 99.9% EGF. Another composition may include about 25% growth factors and about 75% EGF. Alternatively, an example of a composition according to the invention may include major amount of growth factors and a minor amount of EGF, such as about 90% growth factors and about 10% EGF, or about 75% growth factors and about 25% EGF, or about 50% growth factors and 50% EGF.

In addition to growth factors and optionally added plasma or EGF, the composition may also include suitable carriers as well as additional wound repairing and growth promoting active agents, antibacterial agents such as antibiotics and/or bactericides, a fibrinogen component and/or a thrombin component, and pain relievers, as well as one or more vitamin and/or mineral and/or herbal factors that promote wound healing. Examples of suitable agents include safe, efficacious musculoskeletal tissue transplant tissue-like products, such as ALLOMATRIX® by Wright Medical Technology Inc., BIOBRANE® by Bertek Pharmaceuticals Inc., GRAFT JACKET® by Wright Medical Technology Inc., an allograft or allograft-type product (cadaver bone obtained from a tissue bank), or an autograft (the patient's own bone). Alternatively, the compositions of the invention can be used in combination therapy and may be accompanied by the separate administration of additional growth promoting active agents, antibacterial agents, pain relievers, as well as one or more vitamin and/or mineral and/or herbal factors that promote wound healing. Optionally, the composition may additionally contain other additives, such as calcium ions, protease inhibitors, heparin antagonists, substances which promote the infiltration and growth of fibroblasts, such as fibronectin, and a cromolyn compound, a hyaluronic acid, and a corticosteroid. The composition may also be administered in combination with heat and/or moisture therapy.

In embodiments wherein one or more additional growth promoting active agents, one or more antibacterial agents, one or more pain relievers, one or more vitamins, one or more minerals, and/or one or more herbal factors are included, the composition will include an effective amount of the one or more growth factors, an effective amount of the one or more additional growth promoting active agents, an effective amount of the one or more antibacterial agents, an effective amount of the one or more pain relievers, an effective amount of the one or more vitamins, an effective amount of the one or more minerals, and an effective amount of the one or more herbal additives.

Examples of suitable carriers include collagen, such as microcrystalline collagen, oils, aleo vera, a wax, a polyol, one or more fats or oils, one or more emulsifying agents, and/or one or more water-soluble gums and water, optionally also including a preservative. The carrier may be included in the composition in an amount from about 25% to about 50% of the composition, preferably from about 30% to about 40% of the composition.

Examples of suitable additional growth promoting active agents include epidermal growth factors, steroids, enzymes, and hormones, natural (such as having been isolated and purified) or synthetic. The additional growth promoting active agents may be included in the composition in an amount of from about 1% to about 50% of the composition.

Examples of suitable antibacterial agents that may be applied before, during, or after treatment with the composition as a solution or a cream, gel, or a paste, include silver compounds, such as silver nitrate, honey, sulfamylon, silver sulfadiazine neosporin, and a mycin, such as vancomycin, gintamycin, erythromycin or derivative, or a cillin, such as a penicillin, or amoxicillin. The antibacterial agents may be included in the composition in an amount of from about 1% to about 25% of the composition.

Examples of suitable pain relievers and anti inflammatory agents include heparin, bromelain, ozone, analgesics, opioids, and acetaminophen. The pain relievers and anti inflammatory agents may be included in the composition in an amount of from about 1% to about 25% of the composition depending on the type of wound and possibility of infection.

Examples of vitamin factors that may be used in the compositions of the invention or in combination therapy include Vitamin A and/or retinoids, Vitamin E, Vitamin C, Vitamin D, folic acid, vitamin B5, Bromelain, Vitamin B-complex, Zinc (oral and topical), Chondroitin sulfate (topical), Copper, Ornithine alpha-ketoglutarate (OKG), Arginine, Carnosine, chondroitin sulfate (oral), Glucosamine sulfate (oral), icthammol, calamine, silver sulphadiazine, chlorohexadine acetate, coal, tar. The vitamin factors may be included in the composition in an amount of from about 0.1% to about 25% of the composition.

Examples of minerals that may be used in the compositions of the invention include copper, magnesium, manganese, zinc, iron. The mineral factors may be included in the composition in an amount of from about 0.1% to about 25% of the composition.

Examples of herbal factors that may be used in the compositions of the invention include Aloe vera (topical), Chamomile (topical), Gotu kola (oral and topical), Honey (topical), Horse chestnut (topical), Arnica (topical), Bladderwrack (topical), Calendula (topical), Chaparral (topical), Comfrey (topical), Echinacea (topical), Horsetail (oral and topical), Plantain (topical), St. John's wort (topical), Tea tree oil (topical), goldenseal (topical), echinacea (topical), and Witch hazel (topical). The herbal factors may be included in the composition in an amount of from about 1 mg to about 6 mg and make up from about 0.1% to about 25% of the composition.

The amount of the composition that will be applied to a wound of a patient will naturally depend on the type and extent of the wound to be treated. Generally, the amount of the composition that will be applied to a wound can be determined based on a routine visual observation and examination of the extent of the wound. However, the composition will generally be administered to a patient in an effective amount to treat a wound, and the composition will include an effective amount of the growth factors which will be from about 110 ng to about 300 ng. In general, the composition can be applied to a wound in the form of a layer, such as a gel, a cream, or a paste, that is about an Y8 of an inch thick to about ¼ inch thick. The composition may also be applied directly to a wound in a liquid form by aspiration, or it may be applied to a bandage application such as by spraying or pouring it on a bandage, such as gauze, or dressing to be applied to treat a wound. The dressing may be a polyurethane film, a hydrorolloid, or a synthetic skin. Repeated applications of the composition to a wound area may also be required, and will be determined through a routine visual examination of a patient's wound area.

The wound treating compositions of the invention may be prepared by obtaining a growth factor starting material. A growth factor starting material is a material that contains growth factors. Preferably the growth factor starting material is a platelet starting material, such as platelets, platelet rich plasma (PRP), blood, pure platelets, or platelet poor plasma, or combinations of each of these materials, from an autologous or from a single or multi homologous donor(s). Alternatively, the growth factors may be obtained from a growth factor starting material such as bone marrow, breast milk, amniotic fluid, umbilical cord fluid such as cord blood, combinations of each of these types of growth factor starting materials, or from any other tissue from the mammal that the composition is intended to treat. Preferably, the growth factor starting material is in the form of platelet rich plasma (PRP). When the growth factor starting material is PRP, the PRP may, for example, be obtained in 300 ml bags containing 3.3×10¹¹ platelets from any commercially available blood providing source. Preferably, the platelet starting material is pooled from various donors so that, for example, a pool of ten bags of platelets is combined. The concentration of platelets in the platelet starting material may alternatively be in an amount of from about 60,000 platelets/ml to about 1-3 billion platelets/ml, preferably in an amount of about 1 billion to about 2 billion platelets/ml. The platelet starting material may be in a fresh or a non-fresh form, e.g., thawed previously frozen plasma. Thus, as one can appreciate, “obtaining” a growth factor starting material includes a single entity obtaining a growth factor starting material and employing it in the present invention, as well as a party that obtains a growth factor starting material and transfers it to a second party that then employs it in the present invention.

Obtaining the growth factors may generally occur via alternate schemes. For example, the growth factors may be separated from a growth factor starting material by treating the growth factor starting material, such as by a step of a chemically and/or a step of a nonchemically included separation of growth factors from the growth factor starting material. As used herein, a nonchemical separation is to be understood as a procedure that induces release of growth factors from a growth factor starting material by a means other than a normal release mechanism or activation of the growth factor starting material which is generally accomplished by contacting the growth factor starting material with a chemical inducing separation agent such as those described below. A combination of nonchemically induced separation and chemically induced separation can be employed, so that a chemical inducing agent is employed after a nonchemical manipulation of a growth factor starting material. Nonchemical procedures suitable for obtaining growth factors as described above include French press, freeze-thaw, nitrogen cavitation, sonication, heat treatment, hypotonic shock, or lyophilization. Preferably, the growth factors are obtained from the growth factor starting material solely by a nonchemically induced separation. Chemically induced separations, on the other hand, preferably are preformed with suitable chemical inducing separation agents that include thrombin, serotonin, adenosine diphosphate, acetylcholine, and glass or silicon may also be used to initiate a growth factor separation. As used herein, water and salt water are not considered “chemicals” in the context of a chemically induced separation.

An important feature of the present invention is to recover most, preferably substantially all, and most preferably all, of the growth factors that are obtained from the growth factor starting material. In one embodiment, a growth factor starting material is subjected to a nonchemically induced separation, such as lyophilization, without the addition of a fixing agent, such as are used to fix membranes of platelets, and all, substantially all, or at least most, of the growth factors are retained from the growth factor starting material throughout the process. Preferably, at least about 80%, and more preferably at least 90%, of the growth factors in the growth factor starting material are retained and recovered from that material. In addition to retaining all, substantially all, or most, of the growth factors, when the growth factor starting material is a platelet starting material, most, substantially all, and preferably all, of the platelet membranes and other platelet structures are separated from the growth factor starting material. They may then be removed.

In general, the mechanisms of growth factor release include that, while located inside of the platelets, alpha granules will release growth factors through the alpha granule and platelet membrane. Alternatively, alpha granules can be released from platelets after which growth factors are released from the platelets.

The procedure chosen to obtain growth factors from a growth factor starting material, such as platelets, will generally occur at a temperature sufficient and a time sufficient to release growth factors from the growth factor starting material. When a lyophilization procedure is performed, the lyophilization will preferably be at a temperature of from about −20° C. to about −60° C. and for a time of from about 6 hours to about 48 hours, more preferably at a temperature of from about −20° C. to about −50° C. and for a time of from about 12 hours to about 24 hours, and most preferably at a temperature of about −45° C. and for a time of about 24 hours. Generally, however, time is not as precise a parameter as moisture detection is, and the platelet starting material should be lyophilized to a moisture level that approaches or achieves about 0% moisture, such as less than 1% moisture.

Once the growth factor starting material, such as a platelet starting material, has been treated, the resulting product will generally exist as a mixture of growth factors and growth factor starting material remains, such as a mixture of platelet remains and growth factors, or as a mixture of ruptured platelets, alpha granules, and growth factors. The growth factors should then be recovered, and retained, from the treated growth factor containing mixture, preferably along with any other beneficial growth promoting factors that existed in growth factor starting material, and separated from most, substantially all, or all, of platelet membrane remains when the growth factor starting material includes platelets, such as with a platelet starting material. Growth factors may then be recovered by any suitable procedure, such as a separation technique. Separation of growth factors from a mixture may be performed by filtering and treating the mixture with a chemical separation inducing agent as described above, or first treating with a chemical separation inducing agent, and then filtering to obtain a final growth factor composition to be employed as described further herein. Alternatively, the treated mixture may be reconstituted by adding a reconstitution agent such as water, filtered water or distilled water, or saline, or plasma (fresh or non-fresh) to the treated mixture.

The reconstituted mixture may then be subjected to a separation procedure, such as by centrifugation, by a column, or by a filtration system, to separate growth factors from most, and preferably substantially all, of the mixture of growth factors and unwanted elements. For example, in the separation procedure may separate platelet ghosts from most or substantially all of the alpha granules, after which the growth factors may separated from most or substantially all of the alpha granules. Alternatively, when the platelets remain mostly intact, such as when growth factors are extracted without rupturing the platelets, the separation procedure may be used to separate most or substantially all of growth factors from most or substantially all of the intact platelet remains. Centrifugation may be carried out at a speed sufficient and a time sufficient to separate platelet ghosts from alpha granules. Preferably, most or substantially all of the platelet ghosts are separated from most or substantially all of the alpha granules. The lyophilate may first be subjected to a chemical activation of growth factors prior to rehydration, or a chemical activation may occur during or after the rehydration step.

The centrifugation may be carried out at a speed of from about 1,200 rpm to about 5,000 rpm, and for a time of from about 10 minutes to about 1 hour, preferably at a speed of from about 2,000 rpm to about 4,500 rpm, and for a time of from about 15 minutes to about 45 minutes, and most preferably at a speed of from about 2,500 rpm to about 4,000 rpm, and for a time of from about 20 minutes to about 30 minutes. The use of a column or filtration system, in lieu of or in addition to centrifugation, serves to separate most or substantially all of the alpha granules from most or substantially all of the platelet ghosts and collect the alpha granules, or to separate most or substantially all of growth factors from most or substantially all of the intact platelet remains, at the same time. Thus, separation may be by any suitable method, such as by filtration, or where a two phase separated system exists such as in a centrifugation, each method producing a product wherein growth factors are separated from, for example, platelet membrane remains when the growth factor starting material includes platelets.

The resulting separated growth factors are then preferably subjected to a heat sufficient to sterilize the product. Such a heating step may be carried out using a water bath or oven or light source. The heating step will generally be carried out at a temperature sufficient and a time sufficient to sterilize the product. Preferably, the product will be sterilized at a temperature of about 60% and for a time of from about 10 hours to about 12 hours. A heating step will generally also serve to rupture the alpha granules, when they remain present, to produce alpha granule ghosts and growth factors which were contained in the alpha granules. The heating step may occur at any point in the process suitable to sterilize the treated mixture or separated growth factor product.

When sterilized alpha granules, or mixture of alpha granule ghosts and growth factors, remain present, they are then subjected to a separation procedure, such as centrifugation, by a column, or by a filtration system, to separate most or substantially all of the growth factors from the alpha granule ghosts. Centrifugation may be carried out as explained above. As above, a column or filtration system may be used in lieu of or in addition to centrifugation, to separate most or substantially all of the growth factors from the alpha granule ghosts at the same time.

The growth factors obtained may then be nonchemically treated, such as by aliquoting the growth factors into vessels and lyophilizing, either with or without added fresh plasma or previously obtained plasma, and stored. Fresh plasma or previously obtained plasma may be further added to the growth factors obtained and lyophilized to reconstitute the lyophilized mixture. Alternatively, the reconstituting may be accomplished by adding distilled water to the lyophilized mixture. Alternatively, the growth factors obtained need not be lyophilized and can be used directly with or without added fresh plasma or previously obtained plasma.

Thus, in one detailed sequential embodiment, platelets are pooled from, for example, ten 300 ml bags of platelets in plasma, and an amount of the pooled platelet starting material in plasma is filled into one or more vials so that each vial is filled to a level of less than about 50%, such as about 33%, or 25%. So, for example, a plurality of 10 ml vials may each be filled with 3 ml of a pooled plasma starting material. The pooled plasma starting material may be at room temperature, or optionally be subjected to a first freezing step wherein the plasma starting material may be cooled to a temperature no lower than about −50° C., preferably about −40° C.

The platelet starting material may then be lyophilized to freeze and dry the platelet starting material. Generally, the lyophilization device should be set at about −60° C., although the temperature may only reach about −45° C. The lyophilization is complete when the lyophilized material reaches a degree of dryness so that the moisture is minimized to approach about 0% moisture, such as less than 1% moisture.

The lyophilate will generally appear as a round white pellet or cake at the bottom of each vial which is capped or otherwise sealed. The lyophilate is then rehydrated with, for example, water, saline, or fresh plasma, so that each vial-containing lyophilate approaches or achieves its original volume prior to lyophilzation. The vials are then left to stand for about 20 minutes, after which, the contents of each vial is combined. Alternatively, the lyophilates may all be combined and the combined lyophidates are rehydrated so that the original combined volume is approached or achieved. The combined rehydrated product is then subjected to a mixing step, and the mixed product is then centrifuged. The centrifugation may be in a range of from about 1,200 rpm to about 5,000 rpm for a time in a range of from about 15 minutes to about 30 minutes.

After centrifugation, the supernatant is removed and heated to sterilize the supernatant. Generally, sterilization will occur at FDA standards for sterilization which is about 60° C. for 10 hours. After heating, the product is again centrifuged. After centrifugation, the supernatant, which will include water and growth factors, is separated and retained. The product is then subjected to a lyophilization at the same parameters as explained above to produce a lyophilate.

The lyophilate may be stored or immediately rehydrated, such as with water or saline, and preferably with fresh sterilized plasma. Rehydration should be in an amount that approaches or achieves the original volume. Once rehydrated, the mixture should be used within about 8 hours. The lyophilate, or rehydrated lyophilate may then be combined with any of the other additive elements identified above.

In an alternative embodiment, the initial rehydration step, i.e., the rehydration of the lyophilized mixture of platelet ghosts and alpha granules, may be avoided by lyophilizing the platelets at a temperature sufficient and a time sufficient to produce a mixture of platelet ghosts, alpha granule ghosts, and growth factors directly, preferably at a temperature above about −20° C. and a time of about −60° C. Once the platelets and alpha granules have been ruptured, the growth factors may be separated from the mixture and collected, either in a single step or in several steps, such as by is then subjected to a separation procedure, such as centrifugation, by a column, or by a filtration system, to separate the growth factors from most or substantially all of the alpha granule ghosts. The growth factors may then be collected.

In a further embodiment, after the separation step, i.e., the separation of the alpha granules from the rehydrated mixture of platelet ghosts and alpha granules, the alpha granules may be activated, such as by a chemical activation step employing an activator which will bind to the surface of an alpha granule, to trigger the release of growth factors, such as, for example, thrombin. The mixture obtained would then include alpha granules and growth factors, and the growth factors could be separated from the alpha granules by methods as described above. The growth factors may then be lyophilized as described above, i.e., such as by aliquoting the growth factors into vessels and lyophilizing, either with or without added fresh plasma or previously obtained plasma, and stored. Fresh plasma or previously obtained (non-fresh) plasma may be further added to the growth factors obtained and lyophilized to reconstitute the lyophilized mixture. Alternatively, the growth factors obtained need not be lyophilized and can be used directly with or without added fresh plasma or previously obtained plasma. The plasma is preferably present in a non-negligible amount. The growth factors may be collected at any point after they have been separated from the alpha granules.

In another embodiment, the platelet starting material, such as a pooled platelet starting material, may be heated to sterilize the starting material such as at 60° C. for about 10-12 hours. The sterilized material is then subjected to a separation procedure, such as by centrifugation, filtration, or column, etc., and optionally further filtered, and the resultant liquor maybe placed into a vial or on a surgical sponge. The liquor, whether in a vial or on a sponge to be applied to a wound, is then lyophilized as explained above. The lyophilized product may then be stored or rehydrated for immediate use as explained above.

In another embodiment, fresh frozen plasma is lyophilized and the lyophilate is heated. Heating at a low temperature, such as 170 C for 11 minutes to 13 minutes, produces a gel which has easy application qualities. Heating at a high temperature, such as 170 C for 14 minutes to 17 minutes, produces a tissue-like composition which may be formed into a waffer. When subjected to microwave heating for 3 minutes at a high heating level, a tissue-like product is formed. When subjected to microwave heating at the same heat level for 2 minutes and 30 second, a moldable gel was formed. The products obtained have applicability in wound treating. For example, a growth factor powder may be added to one side of the tissue-like waffer and water, or saline, or plasma may be added when the waffer is ready for use.

The heat may be applied in any form (i.e., oven, microwave, steam sterilizer). By varying the heat and or time, the product can be made in a form that ranges from a gel to a strong tissue-like substance. For example, when a vial containing lyophilate is placed in steam sterilizer for 17 minutes at 150° C., the resulting product is a very strong tissue-like substance.

When a vial containing lyophilate is placed in a dry oven for 17 minutes at 150° C. it also produces a very strong tissue-like substance. However, when a vial containing lyophilate is placed in a dry oven for 11 minutes at 150° C. it produces a gel form product.

When subjected to microwave heating for 3 minutes at a high heating level, a tissue-like product is formed. When subjected to microwave heating at the same heat level for 2 minutes and 30 seconds, a moldable gel was formed. The products obtained have applicability in wound treating. For example, the product obtained, whether a moldable gel, tissue-like waffer, powder, or sponge, may then be combined with suitable carriers as well as additional growth promoting active agents, antibacterial agents such as antibiotics and/or bactericides, a fibrinogen component and/or a thrombin component, and pain relievers, as well as one or more vitamin and/or mineral and/or herbal factors that promote wound healing, calcium ions, protease inhibitors, heparin antagonists, and substances which promote the infiltration and growth of fibroblasts, such as fibronectin, and a cromolyn compound, a hyaluronic acid, and a corticosteroid. The composition obtained may be used in forming a patch for application to a wound, such as a dermal patch, or may be applied directly to a wound.

The present invention also provides methods of treating patients, in particular mammals, and most particularly human patients, by administering to a patient in need thereof an effective amount of a composition according to the invention as described above. The effective amount administered will naturally be an amount sufficient to treat the particular type of wound desired to be treated, e.g., burns, cuts, and scrapes, contusions, including oral and otolaryngological wounds, wounds that are caused and treated by plastic surgery, and bone damage, of the body. The method further includes identifying a patient in need of such treatment, and administering successive courses of treatment as necessary. The amount administered, as explained above, will be sufficient to treat a wound in one, or more, application(s), depending on the course of treatment desired.

The present invention also includes a kit for preparing a wound-treating composition. The kit will include the composition, which may be in powder form, sponge form, or tissue-like waffer, and may include instructions for rehydrating the composition, along with a liquid for rehydrating the composition for application to a wound. The kit may contain several separate composition components for repeat applications to a wound. The kit may also include a bandage or a dressing. In addition, the kit may include any of the additive combination elements identified above.

The following Examples describe the preparation of a wound healing composition according to the invention and their use. The inventors are not intended to be limited by the examples disclosed below.

### EXAMPLES

### Example 1

A growth factor composition according to the invention was obtained from platelet rich plasma, obtained from South Texas Blood and Tissue Center. It was supplied in separate 400 ml bags. 25 m is of the PRP were placed in 50 ml vials and lyophilized immediately. The lyophilization was carried out by placing the vials in a lyophilization device (known as a Hull 120) for about 48 hours taking care to not allow a freeze and thaw to occur, not allowing the temperature to freeze below −50° C., and not allowing the temperature to rise above −20° C. Once the lyophilization process is completed the product is then rehydrated and allowed to stand at room temperature for at least 30 minutes. It is then centrifuged (Soval RC3) for 30 min at 500 rpms to cause the platelets to gather at the bottom of the tube. The platelets are then discarded by aspirating the plasma from the tube. The plasma is then placed into a water bath for 10 hours at 60° C., which causes other proteins present to be broken down, with the exception of the cytokines or growth factors. After the heating process, the plasma is placed in the centrifuge and spun again for 30 minutes at 5000 rpms. This will cause all of the destroyed proteins to move to the bottom and removed. The remaining product is then place in single dosage vials, in this case 3 mls of product in a 10 ml vial. The vials are once again lyophilized again as before to obtain the finished product.

### Example 2

PRP was obtained again from South Texas Blood and Tissue Center. The PRP was supplied in 400 ml bags. 25 mls of the PRP was placed in 50 ml vials and lyophilized immediately. Specifically, the vials were placed in lyophilization equipment (Hull 120) for about 48 hours taking care to not allow a freeze and thaw to occur, not allowing the temperature to freeze below −50° C., and not allowing the temperature to rise above −20° C. Once the lyophilization process is completed the product is then rehydrated and allowed to stand at room temperature for at least 30 minutes. It was then centrifuged (Soval RC3) for 30 minutes at 500 rpms to cause the platelets to gather at the bottom of the tube. The platelets are then discarded by aspirating the plasma from the tube. The product is then placed in single use vials (3 mls in 10 ml vials) as before. By not heating this product, it allows the fibrin to stay intact thus allowing the product upon reconstitution, prior to applying to the patient, to add an antagonist to cause the product to con-gel to form a gel for easy application.

### Example 3

A 52 year old male patient with a diabetic ulcer on his left great toe that had been present for 2 years was treated using the composition produced in accordance with Example 1. The composition obtained was rehydrated using 3 mls of deionized water and allowed sit at room temperature for about 30 minutes. It was then place on a dry 4×4 dressing by aspirating the product or pouring the product out of the vial on to a 4×4 dressing. It was then place on the patients wound and then covered with Tegaderm™ made by 3M Health Care. This dressing was allowed to stay in place for at least 4 days. At the end of 4 days the dressing was removed. About 40% wound volume reduction was observed. The wound was cleaned well using normal saline and gauze and then rubbed with a dry gauze to cause bleeding in the wound. The next treatment in the same manner as before was repeated. The treatment was repeated two more times and complete healing was obtained.

### Example 4

A patient having a pressure wound on his left hip in the form of an 8 cm tunnel that had been present for 4 months without healing was treated with the composition. Because of the tunnel form of the wound, the product needed to stick to the tunnel, so the Example 2 product was used. The wound was debrided and cleaned of any necrotic tissue. The product was prepared by adding 3 ml of deionized water and was allowed to sit at room temperature for about 30 minutes. The product was placed into a 10 ml syringe and Thrombin (bovine Mfg. by: GenTrac, Inc) ½ ml (1000 units per ml) and was added to form a gel mixture. The gel was then place into the wound and covered with dry 4×4 dressing and Tegaderm™ and left on for 4 days. On day 4, a fresh dressing was applied and again left for 4 days. This treatment was repeated three more times to achieve complete healing. The total time was 3 weeks.

### Example 5

A horse with a traumatic 15 cm laceration wound on its left hip caused by a barbed wire fence that had been present for 4 days was treated with a composition according to the invention prepared with horse-derived growth factors. Because of the location of the wound, the product needed to stick to the wound, so an Example 2-type product, made from equine PRP, was used. The wound was debrided and cleaned of any dirt and necrotic tissue. The equine product (10 mls) was prepared by adding 10 mls of de-ionized water and allowing it to sit at room temperature for about 30 minutes. The product was placed into a 20 ml syringe and Thrombin (bovine Mfg. by: GenTrac, Inc) ½ ml (1000 units per ml) was added to form a gel mixture. The gel was then place into the wound and covered with dry 4×4 and wrapped with cotton wrap and left on for 4 days. On day 4, a fresh dressing was applied and again left for 4 days. This treatment was repeated four times to achieve complete healing. The total time was 3 weeks.

While the present inventions have been illustrated and described in many embodiments of varying scope, it will at once be apparent to those skilled in the art that variations may be made within the spirit and scope of the inventions. Accordingly, it is intended that the scope of the inventions set forth in the appended claims not be limited by any specific wording in the foregoing description, except as expressly provided.

What is claimed is:

1. A process for obtaining growth factors comprising treating a growth factor starting material nonchemically to release growth factors from the growth factor starting material and recovering growth factors from the treated growth factor starting material.

2. A process according to claim 1 wherein the growth factor starting material is selected from the group consisting of platelets, platelet rich plasma, platelet poor plasma, breast milk, blood, bone marrow, amniotic fluid, umbilical cord fluid, and combinations thereof.

3. A process according to claim 1 wherein the growth factor starting material comprises a platelet starting material.

4. A process according to claim 3 wherein the platelet starting material comprises platelet rich plasma.

5. A process according to claim 1 wherein the nonchemical treatment is selected from the group consisting of freeze-thaw, sonication, lyophilization, French press, nitrogen cavitation, heat treatment, and hypotonic shock.

6. A process according to claim 5 wherein the nonchemcial treatment is lyophilization of the growth factor starting material.

7. A process according claim 6 wherein the growth factor starting material comprises a platelet starting material.

8. A process according claim 7 wherein the platelet starting material comprises platelet rich plasma.

9. A process according to claim 8 further comprising, prior to recovering, reconstituting the lyophilate of the platelet rich plasma.

10. A process according to claim 9 wherein the growth factors are recovered by centrifuging the reconstituted lyophilate.

11. A process according to claim 9 wherein the growth factors are recovered by filtering the reconstituted lypohilate.

12. A process according to claim 1 further comprising heating the recovered growth factors.

13. A process according to claim 9 further comprising heating the reconstitued lyophilate.

14. A process according to claim 10 further comprising heating the centrifuged growth factors.

15. A process according to claim 14 further comprising lyophilizing the heated growth factors.

16. A process according to claim 6 further comprising, prior to recovering, reconstituting the lyophilate of the growth factor starting material.

17. A process according to claim 16 further comprising heating the reconstituted lyophilate.

18. A process according to claim 16 further comprising heating the recovered growth factors.

19. A process according to claim 18 further comprising lyophilizing the heated growth factors.

20. An isolated and purified growth factor composition.

21. An isolated and purified growth factor composition prepared by process claim 15.
